(19) United States
(12) Patent Application Publication
(10) Pub. No.: US 2013/0025003 A1
(43) Pub. Date: Jan. 24, 2013

(54) SORGHUM TRANSFORMATION

(71) Applicants: Anthony Thinh Ngoc Trieu, Oxnard, CA (US); Dongmei Xu, Newbury Park, CA (US)

(72) Inventors: Anthony Thinh Ngoc Trieu, Oxnard, CA (US); Dongmei Xu, Newbury Park, CA (US)

(21) Appl. No.: 13/574,978

(22) PCT Filed: Jan. 27, 2011
(86) PCT No.: PCT/US2011/022738

§ 371 Date: Oct. 4, 2012

Related U.S. Application Data
(60) Provisional application No. 61/299,122, filed on Jan. 28, 2010.

(51) Int. Cl. A01H 5/00; C12N 15/82

(52) U.S. Cl. 800/294; 800/320; 435/469

(57) ABSTRACT

Methods and materials for inducing embryogenic sorghum callus are described. The materials and methods are useful for Agrobacterium-mediated transformation of sorghum.

### TECHNICAL FIELD

This document relates to methods and materials for transformation of sorghum plants. For example, this document provides materials and methods useful for Agrobacterium-mediated transformation of sorghum as well as transformed sorghum plants made by such methods.

### BACKGROUND

Sorghum is a widely grown grain and forage crop that possesses unique attributes, such as adaptability to drought and heat, which can be exploited to support human and animal populations in geographic areas with minimal soil fertility, extreme temperatures, and low precipitation. In addition, sorghum is becoming increasingly important as a feedstock for ethanol production. The ability to introduce, express, and modulate genes in sorghum represents a powerful tool to broaden the germplasm base for plant improvement; however, there is a need for improved transformation procedures for sorghum. For example, sorghum often exhibits a hypersensitive necrotic response to infection with Agrobacterium, making it difficult to maintain transgenic callus and to regenerate transgenic plants. See, e.g. WO/2009/093200 and WO/2009/093201. Moreover, attempts to transform immature sorghum embryos using particle bombardment have met with limited success. See, e.g., Tadesse, et al., Plant Cell Tissue Organ Cult 75, 1-18 (2003). Therefore, improved sorghum transformation methods could facilitate the production of sorghum varieties that exhibit increased resistance to pests, pathogens and environmental stress, and/or possess enhanced nutritional value.

### SUMMARY

This document relates to methods and materials involved in transformation of sorghum plants. For example, methods for transforming sorghum plants via Agrobacterium-mediated transformation as well as transformed sorghum plants are provided herein. Methods and materials described herein can provide more efficient transformation of sorghum, and expand the range of sorghum genotypes that can be transformed.

This disclosure features a method for inducing embryogenic sorghum callus. The method comprising incubating immature sorghum embryos on an induction medium comprising sorbitol and asparagine until embryogenic sorghum callus is formed. The sorbitol can be present at a concentration of about 0.5% w/v to about 4.0% w/v. The asparagine can be present at a concentration of about 50 mg/L to about 1,000 mg/L. The induction medium can further include MS basal salts, myo-inositol, pyridoxine, nicotinic acid, one or more additional amino acids, and an auxin. For example, the induction medium can include MS basal salts, 0.05 mg/L CuSO₄.5 H₂O, 2 mg/L glycine, 200 mg/L asparagine, 100 mg/L cysteine, 100 mg/L myo-inositol, B5 vitamins, 5 mg/L thiamine, 1 mg/L pyridoxine, 1 mg/L nicotinic acid, 2 mg/L 2,4-D, 2.5% sucrose, 1% sorbitol, and 7 g/L agarose, at pH 5.8. The immature sorghum embryos can be obtained from a plant of inbred line B.Tx635, inbred line B.Tx637, inbred line B.Tx627, inbred line B.Tx2752, inbred line BtX430 or inbred line C401.

This disclosure also features a method of making a transformed sorghum cell. The method includes the steps of incubating an immature sorghum embryo on an induction medium comprising sorbitol and asparagine to form an embryogenic sorghum callus; contacting the embryogenic sorghum callus with Agrobacterium in a liquid medium, the Agrobacterium comprising an exogenous nucleic acid whose expression confers resistance to a selection agent; co-cultivating the embryogenic sorghum callus, after the contacting step, on a co-cultivation medium for a period of about 2 to about 5 days; and selecting, on a selection medium, for at least one transformed sorghum cell derived from a co-cultivated embryogenic sorghum callus, the selection medium containing an antibiotic that inhibits the growth of the Agrobacterium and a selection agent that inhibits the growth of untransformed sorghum cells, thereby obtaining the transformed sorghum cell. The selection medium can further include sorbitol and asparagine. The incubating step can include two subculturing periods of 14 days each on the induction medium.

This disclosure also features a method of making a transformed sorghum cell. The method includes the steps of contacting immature sorghum embryos with Agrobacterium on a liquid medium, the Agrobacterium comprising an exogenous nucleic acid whose expression confers resistance to a selection agent; co-cultivating the immature embryos, after the contacting step, on a co-cultivation medium for a period of about 2 to about 5 days, the co-cultivating medium comprising sorbitol and asparagine; and selecting, on a selection medium, for at least one transformed sorghum cell derived from the co-cultivated immature embryos, the selection medium containing an antibiotic that inhibits the growth of the Agrobacterium and a selection agent that inhibits the growth of untransformed sorghum cells, thereby obtaining the transformed sorghum cell.

In some embodiments, the co-cultivating period can be about 3 days. The exogenous nucleic acid can be NPTII and the selection agent paramomycin, or the exogenous nucleic acid can be PAT and the selection agent can be phosphinothricin. In some embodiments, the methods include the step of regenerating at least one transformed sorghum plant from the transformed sorghum cell. The immature sorghum embryos can be from a plant of inbred line B.Tx635, inbred line B.Tx637, inbred line B.Tx627, inbred line B.Tx2752, inbred line B.Tx430 and inbred line C401.

In some embodiments the selecting step can include selecting a plurality of transformed sorghum cells derived from the co-cultivated immature embryos. The methods can further include the step of regenerating a plurality of transformed sorghum plants from the transformed sorghum cells. In some embodiments, the selection medium further comprises one or more auxins. For example, a selection medium can include two auxins (e.g., 2,4-D and NAA).

In any of the methods described herein, transformed sorghum cells can be incubated in a resting medium for about 7 to about 14 days before regenerating transformed sorghum plants. A resting medium can include asparagine, cysteine, an auxin, a cytokinin, and sorbitol. In one embodiment, a resting medium includes MS basal salts, 0.05 mg/L CuSO₄.5 H2O, 2 mg/L glycine, 200 mg/L asparagine, 100 mg/L cysteine, 100 mg/L myo-inositol, B5 vitamins, 5 mg/L thiamine, 1 mg/L pyridoxine, 1 mg/L nicotinic acid, 0.5 mg/L NAA, 2.5% sucrose, 1% sorbitol, and 7 g/L agarose, at pH 5.8.

Unless otherwise defined, all technical and scientific terms used herein have the same meaning as commonly understood by one of ordinary skill in the art to which the invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting. Other features and advantages of the invention will be apparent from the following detailed description. Applicants reserve the right to alternatively claim any disclosed invention using the transitional phrase “comprising,” “consisting essentially of,” or “consisting of,” according to standard practice in patent law.

### DETAILED DESCRIPTION

This document relates to methods and materials involved in transformation of sorghum, via Agrobacterium-mediated transformation of embryogenic sorghum cells. It has been discovered that embryogenic sorghum cells are more readily produced when sorbitol and/or asparagine are included in the induction medium. Media are provided herein that are suitable for embryogenic sorghum callus induction, co-cultivation of embryogenic sorghum calli with Agrobacterium, selection of transformed cells, and regeneration of transformed sorghum plants.

### I. SORGHUM TISSUE CULTURE

In general, transformation of sorghum comprises Agrobacterium-mediated transformation of embryogenic cells in culture and selection of transformed cells in culture. The methods described herein can comprise several stages. For example, transformation can include inducing embryogenic calli from plant tissue, preparing Agrobacterium transformants, infecting embryogenic calli with prepared Agrobacterium, selecting transformed calli, regenerating a plant from transformed cells, and rooting the regenerated plant. Each stage is described in more detail below.

The methods described herein involve the use of liquid or semi-solid medium. A semi-solid medium can be, for example, Murashige and Skoog (MS) medium containing agar and a suitable concentration of an auxin and a suitable concentration of a cytokinin. In some cases, plant tissue can be placed directly onto the medium or can be placed onto a filter that is then placed in contact with the medium.

A. Immature Embryos

Immature embryos are an intact tissue that is capable of cell division to give rise to callus cells that can then differentiate to produce tissues and organs of a whole plant. Immature embryos can be obtained from immature inflorescences of a fertilized sorghum plant. Methods for isolating immature embryos from sorghum are described in Cao et al., Plant Cell Tiss. Org. Cult., 20:101-110 (1990) and Gao et al., Plant Biotech. J., 3:591-599 (2005) Immature embryos are aseptically isolated from the developing kernel and held in sterile medium until use. The immature embryos are typically isolated approximately 9 days to about 17 days after pollination, e.g., about 9 days to about 16 days after pollination, or about 10 days to about 15 days after pollination. The immature embryos typically are from about 0.6 to about 1.8 mm in length, e.g., about 0.6 to about 1.2 mm, or about 0.8 to about 1.6 mm.

Immature embryos can be used directly for Agrobacterium-medium transformation, by culturing for several days, generally about 3 to about 10 days, preferably about 5 to about 8 days, prior to infection with Agrobacterium. Alternatively, immature embryos can be used to generate embryogenic callus, which is then used for Agrobacterium-medium transformation.

B. Embryogenic Callus

Generally, embryogenic callus is derived from immature tissue such as immature embryos, immature inflorescences, and the basal portion of young leaves. Alternatively, the callus can be originated from anthers, microspores, or mature embryos. A useful tissue for producing regenerable callus is the scutellum of immature sorghum embryos. Embryogenic calli can be generated from plant tissue harvested from in vitro, greenhouse, or field-grown plants.

The prepared explant is cultured on a semi-solid induction medium to generate embryogenic callus. Callus induction media comprises a basal medium, micronutrients and vitamins, amino acids, an auxin, sugar or sugar alcohol, and a gelling agent. One of the amino acids present in the induction medium is asparagine, at a concentration of from about 50 mg/L to about 1,000 mg/L, e.g., 50 to 500 mg/L, 100 to 500 mg/L, 100 to 300 mg/L, 150 to 300 mg/L, 100 to 700 mg/L, or 150 to 250 mg/L. One of the sugar or sugar alcohols present in the induction medium is sorbitol, at a concentration (w/v) of 0.5% to 4.0%, e.g., 0.5% to 1.0%, 0.5% to 2.0%, 0.7% to 1.0%, 1.0% to 2.5%, 1.0% to 1.5%, or 0.7% to 3.0%.

Typically, the basal medium is 1×MS, although N6, NB, or Gamborg B-5 basal media are also suitable. An induction medium typically also includes amino acids in addition to asparagine, e.g., glycine, cysteine, and/or casamino acids. An induction medium typically also includes sugars or sugar alcohols in addition to sorbitol, e.g., sucrose or maltose. An induction medium also includes micronutrients and vitamins such as B5 vitamins, myo-inositol, thiamine HCl, pyridoxine HCl, ascorbic acid, CuSO₄.5H₂O, and nicotinic acid. Induction medium is typically semi-solid and thus includes a gelling agent and has a pH of about 5.7 to 5.9.

An induction medium also includes an auxin, but typically does not include a cytokinin. Suitable auxins include indole-3-acetic acid (IAA), 1-naphthaleneacetic acid (NAA), indole-3-butryic acid (IBA), 2-methoxy-3,6-dichlorobenzoic acid (dicamba), and 2,4-D.

The period of time that the plant tissue is cultured in induction media, the number of days until calli form, the temperature for incubation of the plant tissue, the light conditions can vary to some extent while still maintaining the ability to form embryogenic callus. In some cases, callus induction can include culturing prepared tissue for about 2, 3, 4, or 5 weeks. Calli can be subcultured on induction media for 1, 2, 3 or more subcultures. Calli can be subcultured on induction media every 2-3 weeks, for example, in order to expand the amount of callus tissue available for transformation. At each subculture, the tissue is observed under a dissecting microscope and friable or semi-compact non-mucilaginous tissue is chosen for transfer to fresh media. For those sorghum lines that produce pigment, calli can be subcultured more frequently, if desired, in order to reduce the amount of pigment present when the calli are contacted with Agrobacterium.

Portions of cultured calli can be transferred to regeneration media in order to determine the regeneration capacity and thus verify that the callus is embryogenic. The regeneration capacity can be estimated by counting the number of regenerated plantlets per petri dish.

The use of sorbitol and asparagine improves callus induction efficiency by about 20 to 32%, as determined by comparing the number of calli formed in the presence of sorbitol and asparagine to the number of calli formed in an induction medium that does not contain sorbitol and asparagine. The increase in relative callus induction efficiency can be up to 40%.

C. Sorghum Lines

Sorghum plants suitable for obtaining recipient tissue for transformation and for generating embryogenic callus include plants of Sorghum bicolor inbred lines B.Tx635; B.Tx637; B.Tx627; B.Tx2752; B.Tx430, Wheatland, and C401. Also suitable are plants of Sorghum bicolor hybrids such as Pioneer Hi-Bred® 31G65 (RR2) and DeKalb® DK-40Y. Also suitable are plants of Sorghum bicolor ssp. sudanense L. (Sorghum×drummondii). It is contemplated that plants of Sorghum×sudangrass hybrids (Sorghum bicolor×S. bicolor spp. sudanese) and Sorghum×almum hybrids may also be suitable.

### II AGROBACTERIUM-MEDIATED TRANSFORMATION

Generally, sorghum transformation includes contacting sorghum cells with Agrobacterium for a period of time, followed by co-cultivating the infected cells for a period of time. The contacting and co-cultivating steps generally take place in a liquid medium.

A number of Agrobacterium strains are suitable for use with the methods described herein. For example, Agrobacterium tumefaciens strain C58, LBA4404, EHA101, EHA105, or EHA109 can be used to produce a transformed sorghum plant. In some cases, a strain of Agrobacterium rhizogenes can be used. In preparation for inoculation of embryogenic calli, an overnight culture of Agrobacterium typically is resuspended in co-cultivation medium and the bacterial concentration is adjusted to an optical density of about 0.3 to 0.5 at OD₆₀₀. The culture can then be incubated for a short period of time in the dark at 22 to 24° C., while shaking at about 150 rpm, until ready for carrying out the contacting step.

A. Contact

Sorghum cells are contacted with the Agrobacterium suspension in a liquid co-cultivation medium for a short period of time, e.g., about 15 to about 60 minutes, about 20 to about 40, about 25 to about 35, or about 30 minutes. The contacting typically is carried out in the dark with gentle shaking at room temp. In some instances, the sorghum cells are heat-shocked (incubated at about 40-45° C. for about 1-3 minutes) and cooled to ambient temperature before contacting with Agrobacterium.

The number of Agrobacterium included in the contacting step is typically about 5 ml to 100 ml of an Agrobacterium suspension having an optical density of about 0.3 to 0.5. The amount of sorghum cells included in the contacting step is typically about 5 to 100 gm fresh weight embryogenic callus or immature embryos.

A co-cultivation medium for use in transforming sorghum cells includes a basal medium, vitamins and micronutrients, sugars, amino acids, an auxin, a cytokinin, and an activator of Agrobacterium virulence genes, at a pH of 5.3 to 5.5. Suitable vitamins and micronutrients include 1/10× B5-vitamins and ascorbic acid at 5 to 15 mg/L. Suitable amino acids include L-cysteine and L-glutamine, although casamino acids typically are not included. Suitable sugars include maltose and glucose. A suitable auxin is 2,4-D and a suitable cytokinin is 6-benzyl aminopurine, also known as benzyladenine (BA), at a ratio by weight of about 4 to 1. Other suitable cytokinins include kinetin, zeatin, adenosine phosphate, and thidiazuron (TDZ). A suitable Agrobacterium activator is acetosyringone.

For example, a suitable co-cultivation medium contains 1/10 MS basal medium, B5 vitamins, about 1 to 5% maltose, about 0.5 to 2% glucose, about 5 to 15 mg/L ascorbic acid, about 100 to 300 mg/L L-cysteine, about 10 to 60 mg/L L-glutamine, about 1 to 4 mg/L 2,4-D, about 0.20 to 1 mg/L BA, and about 100 to 400 μM acetosyringone, at a pH of 5.4. Specific examples of co-cultivation media are set forth in Tables 1 and 4.

B. Co-Cultivation

After contacting, excess liquid is removed from the sorghum tissue. The sorghum tissue is then transferred to filter paper moistened with co-cultivation medium and incubated at 22° C. to about 26° C. It is preferable to carry out co-cultivation on filter paper that has not been saturated with co-cultivation medium. A suitable amount of medium is from about 2.0 to about 2.3 ml of co-cultivation medium per 70 mm diameter Whatman #1 filter paper. The duration of co-cultivation is for about 2-6 days, e.g., about 2, 3, 4, 5, or 6 days. Cells are co-cultivated in the dark.

C. Selection/Screening

Following co-cultivation, excess liquid is removed from the sorghum tissue, for example, by rinsing the tissue with water and blotting briefly with sterile tissue paper. The tissue is then transferred to a selection medium and incubated in the dark for several days, e.g., about 6 days to about 14 days, at 25° C. to about 29° C.

A selection medium typically is semi-solid, has a pH of about 5.6 to 5.9, and includes a basal medium, nutrients, micronutrients and vitamins, amino acids, an auxin, sugar and/or sugar alcohols, and a gelling agent. An auxin such as Dicamba is particularly useful in a selection medium when transforming sweet sorghum varieties, lines, or hybrids. In some embodiments, a selection medium contains at least two auxins (e.g., 2,4-D and NAA). Selection medium also includes a selection agent that inhibits the growth of non-transformed cells such that selection for transformed cells can occur, i.e., cells that are resistant to the inhibitory effects of a selection agent in the selection medium grow a faster rate than non-transformed cells on such media. Resistance to the selection agent indicates successful transfer and expression of the selectable marker construct in such cells. The selection agent is chosen based on the selectable marker present on the exogenous nucleic acid transferred by Agrobacterium. Kanamycin, neomycin, paromomycin, butirosin, gentamycin B or geneticin are suitable selection agents when the exogenous nucleic acid comprises a selectable marker encoding an NPTII polypeptide. For example, a selection medium can comprise about 40 to 120 mg/L paromomycin, e.g., 50 mg/L or 100 mg/L paromomycin. Phosphinothricin is a suitable selection agent when the exogenous nucleic acid comprises a selectable marker encoding a phosphinothricin acetyl transferase (PAT) polypeptide. Selection agents suitable for use with other selectable markers are known.

A selection medium also includes an antibiotic for inhibiting the growth of any residual Agrobacterium that have been transferred along with sorghum cells. Such antibiotics include, without limitation, Timentin® (ticarcillin disodium and clavulanate potassium), cefotaxime, carbenicillin, and clavamox (amoxicillin and lithium clavulanate).

A suitable selection medium for use when an NPTII selectable marker is used comprises MS basal salts and B5 vitamins, about 0.0125 to 2.0 mg/L CuSO₄.5H₂O, about 0.50 to 10 mg/L glycine, about 50 to 500 mg/L myo-inositol, about 0.1 to 10 mg/L thiamine HCl, about 0.5 to 5 mg/L pyridoxine HCl, about 0.5 to 5 mg/L nicotinic acid, about 0.5 to 3 gm/L casamino acid, about 1 to 3 mg/L 2,4-D, about 1 to 3% sucrose, about 0.5% to about 3% sorbitol, about 20 to 500 mg/L asparagine, about 100 to 200 mg/L carbenicillin, about 40 to 100 mg/L paramomycin or Timentin®, and about 5 to 10 g/L agarose. Such a selection medium further can include 0.3 to 0.6 mg/L NAA. Tables 1 and 4 provide specific examples of selection media.

The conditions for selection of transformed cells can be modified to some extent while still maintaining the ability select stably transformed cells capable of regeneration. For example, tissue may be subcultured on fresh selection media, i.e., one, two, or more times, and the concentration of the selection agent modified at each subculture. Thus, when NPTII is the selectable marker, tissue can be cultured on a first selection medium comprising about 40 to 50 mg/L paromomycin for about 5 to 10 days and then cultured on a second selection medium comprising about 100 mg/L paromomycin for about 2, 3, or 4 weeks.

For embryogenic callus, the relative transformation efficiency of the methods described herein ranges from about 1% up to about 70%, determined by dividing the number of stably transformed calli by the total number of calli subjected to the co-cultivation. For example, the transformation efficiency can be from about 1% to about 20%, about 1% to about 40%, about 20% to about 40%, about 40% to about 60%, or about 40% to about 70% using the methods and compositions described herein.

For immature embryos, the relative transformation efficiency of the methods described herein is contemplated to be from about 0.3% up to about 3.0%, determined by dividing the number of stably transformed immature embryo-derived calli by the total number of immature embryo-derived tissue pieces subjected to the co-cultivation. For example, the transformation efficiency can be 0.3, 1.0, or 3.0% using the methods and compositions described herein. Typically, the relative transformation efficiency for immature embryos is between about 0.3 and 4.0%.

D. Regeneration

Following selection of transformed cells, sorghum plants are regenerated from such cells on regeneration medium. In some embodiments, the transformed cells are transferred to a resting medium for about seven to fourteen days before regeneration. A resting medium typically includes a basal medium, nutrients, micronutrients and vitamins, amino acids (e.g. one or more of asparagine, cysteine, and glycine), an auxin (e.g., NAA), sugar and/or sugar alcohols (e.g., sorbitol), and a gelling agent. A specific example of a resting medium is set forth in Table 4.

Selected callus tissue can be transferred to semi-solid regeneration medium to form shoots, and such shoots can be transferred to a semi-solid rooting medium to form plantlets. Methods for sorghum regeneration are reported in Kamo et al., Bot. Gaz 146:327-334 (1985), West et al., Plant Cell 5:1361-1369 (1993), and Duncan et al., Planta 165:322-332 (1985). In some cases, the selection agent is incorporated into the media to minimize or eliminate the regeneration of any non-transformed plants that may have survived the selection.

i. Shoot Formation

Transformed sorghum cells are transferred to a semi-solid regeneration medium to allow for shoot formation. Regeneration medium typically comprises a basal medium, vitamins, an auxin and a cytokinin, sugars, amino acids, an antibiotic active against Agrobacterium and a gelling agent. See Tables 1 and 4 for specific examples of regeneration media. For example, a regeneration medium can comprise 1×MS or N6 basal salts, 1× B5 vitamins, about 0.5 to 3 mg/L BA, about 0.05 to 0.3 mg/L NAA, about 0.5 to 2% maltose, about 25 to 100 mg/L L-glutamine, about 1 to 3% sucrose, about 6 to 8 g/L agar, about 80 to 120 mg/L paromomycin, and about 75 to 200 mg/L carbenicillin, at a pH of 5.7 to 5.9. The ratio of cytokinin to auxin in regeneration medium is usually about 10:1. A suitable regeneration medium is 1×MS basal salts, 50 mg/L L-glutamine, 1× B5 vitamins, 2 mg/L BA, 0.2 mg/L NAA, 1% maltose, 2% sucrose, 7 g/L agar, 100 mg/L paromomycin and 125 mg/L carbenicillin, at a pH of 5.8.

Cells are cultured on regeneration medium for 3 to 4 weeks at 28° C. in a growth chamber under a 16/8 hr day/night photoperiod. However, culture conditions can be varied to some extent while still maintaining the ability to produce shoots. For example, cells can be cultured for about 2, 3, 4, 5, or 6 weeks, and/or the temperature can be 25, 26, 27, or 28° C.

ii. Root Formation

Shoot that have formed are transferred to a semi-solid rooting media. Rooting media typically comprises a basal medium, amino acids, micronutrients and vitamins, sugars, an auxin, and a gelling agent. Rooting media typically also contains an antibiotic that inhibits the growth of Agrobacterium. See Tables 1 and 4 for specific examples of rooting media. For example, a rooting media can comprise about ½ to 1×MS basal salts, about 0.1 to 0.3 mg/L glycine, about 0.10 to 1 mg/L thiamine, about 0.50 to 2 mg/L pyridoxine, about 0.50 to 2 mg/L nicotinic acid, about 50 to 200 mg/L myo-inositol, about 1 to 2% sucrose, about 0.5 to 1 mg/L NAA, about 75 to 200 mg/L carbenicillin, and about 3 to 36 g/L agarose, at a pH of 5.7 to 5.9. A suitable rooting media is ½ MS basal salts, 0.2 mg/L glycine, 0.5 mg/L thiamine, 1 mg/L pyridoxine, 1 mg/L nicotinic acid, 100 mg/L myo-inositol, 1.5% sucrose, 0.5 mg/L NAA, 125 mg/L carbenicillin, and about 5 g/L agarose, at a pH of 5.8.

Cells are cultured on rooting medium for 3 to 4 weeks at 28° C. in a growth chamber under a 16/8 hr day/night photoperiod. However, culture conditions can be varied to some extent while still maintaining the ability to produce plantlets. For example, shoots can be cultured for about 5 or 6 weeks, and/or the temperature can be 26, 27, or 28° C.

### III EXOGENOUS NUCLEIC ACIDS

The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

Agrobacterium strains used in the methods described herein contain an exogenous nucleic acid for transfer to sorghum cells, where the exogenous nucleic acids typically become stably integrated into genomic DNA. An exogenous nucleic acid integrated into a plant genome is flanked by one or both of the right and/or left T-DNA border sequences, 25-base-pair sequences of imperfect direct repeats that are required for transfer from Agrobacterium to a plant genome. A preselected exogenous nucleic acid is typically inserted in a recombinant nucleic acid construct, between a right T-DNA border sequence and a left T-DNA border sequence using standard molecular biological techniques. In some cases, a recombinant nucleic acid construct for use with the methods described herein can include functional plant analogs of the Agrobacterium T-DNA borders. For example, plant transfer DNA sequences have been described in Rommens et al. (“Crop Improvement through Modification of the Plant's Own Genome,” Plant Physiology, 135: 421-431, 2004).

An exogenous nucleic acid can be a naturally occurring nucleic acid that is not immediately contiguous with both of the sequences with which it is immediately contiguous (one on the 5′ end and one on the 3′ end) in the naturally occurring genome of the organism from which it is derived. For example, an exogenous nucleic acid molecule can be an isolated DNA molecule that is incorporated into a vector, an autonomously replicating plasmid, or into the genomic DNA of a prokaryote or eukaryote. In addition, an exogenous nucleic acid can include a nucleic acid molecule that is part of a hybrid or fusion nucleic acid sequence. An exogenous nucleic acid can include sequences encoding polypeptides, or transcription factors originating from various species, including, but not limited to, plants, algae, fungi, monera, bacteria, archaeobacteria, protista, and animals.

An exogenous nucleic acid can be a recombinant nucleic acid construct including origins of replication, scaffold attachment regions (SARs), and/or markers. For example, a recombinant nucleic acid construct can include a marker for selection of Agrobacterium transformants. In some cases, a recombinant nucleic construct can be the shuttle vector of a binary vector system or a superbinary vector (e.g., pBin19, pBI121, pCAMBIA series, pPZP series, pGreen series, pGA482, pSB11e, pSB1e, pPCV001, pCLD04541, pBIBAC series, and pYLTAC series as described in Komori et al., “Current Status of Binary Vectors and Superbinary Vectors,” Plant Physiology 145:1155-1160 (2007)).

An exogenous nucleic acid can include a nucleotide sequence encoding a screenable marker for conferring a screenable phenotype on a transformed plant cell. A screenable marker can be used to detect the presence of a transferred exogenous nucleic acid, or measure the expression level of a transferred exogenous nucleic acid in a transformed plant cell or tissue. A screenable marker can be used to assay transient expression. Suitable screenable markers can include a beta-glucuronidase (GUS) polypeptide, a luciferase polypeptide, and a Green Fluorescent Protein (GFP) polypeptide.

Selectable Marker

At least one of the exogenous nucleic acids with which Agrobacterium is transformed comprises a nucleotide sequence that, when expressed, confers a selectable phenotype on a plant cell. Expression of a selectable marker allows for preferential selection of stably transformed cells, tissues and/or plants, in the presence of a selection agent. In some cases, a selectable marker confers resistance to an antibiotic (e.g., kanamycin, paromomycin, or hygromycin), or an anti-neoplastic agent (e.g., methotrexate).

In some embodiments, a selectable marker is a polypeptide that confers herbicide resistance on plants expressing the polypeptide, e.g., bromoxynil, chlorosulfuron or phosphinothricin resistance. Herbicide resistance can be herbicide tolerance, such as tolerance to glyphosate and bromoxynil. Polypeptides conferring resistance to a herbicide that inhibits the growing point or meristem, such as an imidazolinone or a sulfonylurea can be suitable. Exemplary polypeptides in this category can be mutant acetohydroxy acid synthase (AHAS) enzymes as described, for example, in U.S. Pat. Nos. 5,767,366 and 5,928,937. U.S. Pat. Nos. 4,761,373 and 5,013,659 are directed to plants resistant to various imidazolinone or sulfonamide herbicides. U.S. Pat. No. 4,975,374 relates to plant cells and plants containing a gene encoding a mutant glutamine synthetase (GS) resistant to inhibition by herbicides that are known to inhibit GS, e.g. phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides for resistance to glyphosate are also suitable. Typically, glyphosate resistance is conferred by an altered 5-enolpyruvyl-3-phosphoshikimate (EPSP) synthase or a glyphosate oxidoreducatase polypeptide. See, for example, U.S. Pat. No. 4,940,835 and U.S. Pat. No. 4,769,061. Such polypeptides can confer resistance to glyphosate herbicidal compositions, including without limitation glyphosate salts such as the trimethylsulphonium salt, the isopropylamine salt, the sodium salt, the potassium salt and the ammonium salt (see e.g., U.S. Pat. Nos. 6,451,735 and 6,451,732). Polypeptides for resistance to phosphono compounds such as glufosinate ammonium or phosphinothricin, and pyridinoxy or phenoxy propionic acids and cyclohexones are also suitable (see e.g., European application No. 0 242 246, U.S. Pat. Nos. 5,879,903, 5,276,268 and 5,561,236). A polypeptide can confer resistance to herbicides such as triazines and benzonitriles that inhibit photosynthesis, e.g., U.S. Pat. No. 4,810,648, or herbicides such as 2,2-dichloropropionic acid, sethoxydim, haloxyfop, imidazolinone herbicides, sulfonylurea herbicides, triazolopyrimidine herbicides, s-triazine herbicides and bromoxynil. Also suitable is a herbicide-resistant polypeptide such as a protox enzyme (see e.g., U.S. Patent Application No. 20010016956, and U.S. Pat. No. 6,084,155).

Sequences of Interest

A number of other nucleic acids can be introduced into sorghum by the methods described herein. Sequences of interest that can be used in the methods described herein include, but are not limited to, sequences encoding genes or fragments thereof that modulate cold tolerance, frost tolerance, heat tolerance, drought tolerance, water used efficiency, nitrogen use efficiency, pest resistance, biomass, chemical composition, plant architecture, and/or biofuel conversion properties. In particular, exemplary sequences are described in the following applications which are incorporated herein by reference in their entirety: US20080131581, US20080072340, US20070277269, US20070214517, US 20070192907, US 20070174936, US 20070101460, US 20070094750, US20070083953, US 20070061914, US20070039067, US20070006346, US20070006345, US20060294622, US20060195943, US20060168696, US20060150285, US20060143729, US20060134786, US20060112454, US20060057724, US20060010518, US20050229270, US20050223434, and US20030217388.

A nucleotide sequence encoding a polypeptide or transcription product that affects abiotic stress resistance, including, but not limited to flowering, seed development, enhancement of nitrogen utilization efficiency, altered nitrogen responsiveness, drought resistance or tolerance, cold resistance or tolerance, and salt resistance or tolerance, and increased yield under stress, can be introduced into Sorghum by the methods described herein (see e.g., PCT Application WO/00/73475 (modulating water-use efficiency through alteration of malate); U.S. Pat. No. 5,892,009, U.S. Pat. No. 5,965,705, U.S. Pat. No. 5,929,305, U.S. Pat. No. 5,891,859, U.S. Pat. No. 6,417,428, U.S. Pat. No. 6,664,446, U.S. Pat. No. 6,706,866, U.S. Pat. No. 6,717,034, U.S. Pat. No. 6,801,104, and PCT Applications WO/2000/060089, WO/2001/026459, WO/2001/035725, WO/2001/034726, WO/2001/035727, WO/2001/036444, WO/2001/036597, WO/2001/036598, WO/2002/015675, WO/2002/017430, WO/2002/077185, WO/2002/079403, WO/2003/013227, WO/2003/013228, WO/2003/014327, WO/2004/031349, WO/2004/076638, WO/98/09521, and WO/99/38977 (mitigating negative effects of freezing, high salinity, and drought on plants, as well as conferring other positive effects on plant phenotype); US 2004/0148654 and WO/01/36596 (improving agronomic phenotypes such as increased yield and/or increased tolerance to abiotic stress); PCT Applications WO/2000/006341 and WO/04/090143, U.S. application Ser. No. 10/817,483 and U.S. Pat. No. 6,992,237 (modulating cytokinin expression to increase stress tolerance, such as drought tolerance, and/or increased yield); also see PCT Applications WO/02/02776, WO/2003/052063, JP2002281975, U.S. Pat. No. 6,084,153, WO/01/64898, U.S. Pat. Nos. 6,177,275 and 6,107,547 (enhancement of nitrogen utilization and altered nitrogen responsiveness); and see, US 20040128719, US 20030166197, PCT Application WO/2000/32761 (ethylene alteration); and see, US 20040098764 or US 20040078852 (plant transcription factors or transcriptional regulators of abiotic stress)). Other polypeptides and transcription factors that affect plant growth and agronomic traits such as yield, flowering, plant growth and/or plant structure, can be introduced or introgressed into sorghum plants (see e.g., PCT Application WO/97/49811 (LHY), WO/98/56918 (ESD4), WO/97/10339 and U.S. Pat. No. 6,573,430 (TFL), U.S. Pat. No. 6,713,663 (FT), WO/96/14414 (CON), WO/96/38560, WO/01/21822 (VRN1), WO/00/44918 (VRN2), WO/99/49064 (GI), WO/00/46358 (FRI), WO/97/29123, U.S. Pat. No. 6,794,560, U.S. Pat. No. 6,307,126 (GAI), WO/99/09174 (D8 and Rht), and WO/2004/076638 and WO/2004/031349 (transcription factors)).

An exogenous nucleic acid can include a nucleotide sequence that results in male-sterility, e.g., pollen is either not formed or is nonviable. Suitable male-sterility systems include cytoplasmic male sterility (CMS), nuclear male sterility, and molecular male sterility. For example, an exogenous nucleic acid can be a transgene that inhibits microsporogenesis and/or pollen formation. A number of different methods of conferring male sterility are available. For example, multiple mutant genes or expression of transgenes can be used to silence one or more nucleic acid sequences necessary for male fertility (see e.g., U.S. Pat. Nos. 4,654,465, 4,727,219, and 5,432,068, EPO Publication No. 329, 308 and PCT Application WO/90/08828). In some cases, infertility can be achieved using a recombinant nucleic acid construct comprising a sequence encoding a deacetylase polypeptide, under the control of a tapetum-specific promoter, with the application of N-acetyl-phosphinothricin (see e.g., PCT Application WO/01/29237), various stamen-specific promoters (see e.g., PCT Applications WO/92/13956 and WO/92/13957) or using a barnase/barstar transgene system (see e.g., Paul et al., Plant Mol. Biol. 19:611-622 (1992)).

Additional examples of nuclear male and female sterility systems and genes are described in U.S. Pat. No. 5,859,341; U.S. Pat. No. 6,297,426; U.S. Pat. No. 5,478,369; U.S. Pat. No. 5,824,524; U.S. Pat. No. 5,850,014; and U.S. Pat. No. 6,265,640.

iii. Regulatory Regions

The choice of regulatory regions to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a nucleic acid can be modulated in a similar manner.

Some suitable regulatory regions initiate transcription only, or predominantly, in certain cell types. Methods for identifying and characterizing regulatory regions in plant genomic DNA are known, including, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of regulatory regions are described below. Some of the regulatory regions indicated below as well as additional regulatory regions are described in more detail in U.S. Patent Application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/23639; PCT/US05/034308; PCT/US05/034343; and PCT/US06/038236; PCT/US06/040572; and PCT/US07/62762. Sequences of regulatory regions are also set forth in the sequence listings of PCT/US06/040572; PCT/US05/034343; U.S. patent application Ser. No. 11/172,703; PCT/US07/62762; and PCT/US06/038236.

It will be appreciated that a regulatory region may meet criteria for one classification based on its activity in one plant species, and yet meet criteria for a different classification based on its activity in another plant species.

A promoter is considered broadly expressing when it promotes transcription in all or most tissues, in more than one, but not necessarily in all, cell types within all tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds.

Root-active promoters confer transcription in root tissue, e.g., root endodermis, root epidermis, or root vascular tissues. In some embodiments, root-active promoters are root-preferential promoters, i.e., confer transcription only or predominantly in root tissue.

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used.

Promoters that are active in ovary tissues such as the ovule wall and mesocarp can also be useful, e.g., a polygalacturonidase promoter, the banana TRX promoter, the melon actin promoter, YP0396, and PT0623.

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable.

Promoters active in photosynthetic tissue confer transcription in green tissues such as leaves and stems. Most suitable are promoters that drive expression only or predominantly in such tissues.

Promoters that have high or preferential activity in vascular bundles may also be useful, such as the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible promoters confer transcription in response to external stimuli such as chemical agents or environmental stimuli. For example, inducible promoters can confer transcription in response to hormones such as giberellic acid or ethylene, or in response to light or drought. Examples of nitrogen-inducible promoters include PT0863, PT0829, PT0665, and PT0886. Examples of shade-inducible promoters include PRO924 and PT0678. An example of a promoter induced by salt is rd29A (Kasuga et al. (1999) Nature Biotech 17: 287-291).

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

A stem promoter may be specific to one or more stem tissues or specific to stem and other plant parts. Stem promoters may have high or preferential activity in, for example, epidermis and cortex, vascular cambium, procambium, or xylem.

Reproductive tissue promoters are regulatory sequences that drive expression primarily in, but are not necessarily exclusive to, tissues that are required for plant sexual reproduction. These tissues include, but are not limited to, inflorescence meristem, floral meristem, floral organs, and cells of the gametophyte.

Other classes of promoters include, but are not limited to, shoot-preferential, callus-preferential, trichome cell-preferential, guard cell-preferential such as PT0678, tuber-preferential, parenchyma cell-preferential, and senescence-preferential promoters.

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, for example, more than one regulatory region can be operably linked to the sequence of a polynucleotide encoding a biomass-modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### IV. SORGHUM PLANTS

Transgenic sorghum plants and cells comprising at least one exogenous nucleic acid are described herein. A transgenic sorghum plant or cell contains at least one Agrobacterium T-DNA border sequence, or a functional analogue of a T-DNA border from a plant, e.g. a P-DNA border as described in Rommens et al., Plant Physiology, 139: 1338-1349 (2005).

A sorghum plant or cell can be transformed by having a construct integrated into its genome, i.e., can be stably transformed. Stably transformed cells typically retain the introduced nucleic acid with each cell division. A sorghum plant or cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions.

Transgenic sorghum cells can constitute part or all of a whole sorghum plant. Such plants can be grown in a manner suitable for sorghum, either in a growth chamber, a greenhouse, or in a field. Transgenic sorghum plants can be bred as desired for a particular purpose, e.g., to introduce a recombinant nucleic acid into other lines, to transfer a recombinant nucleic acid to other species, or for further selection of other desirable traits. As used herein, a transgenic sorghum plant also refers to progeny of an initial transgenic plant provided the progeny inherits the transgene. Seeds produced by a transgenic plant can be grown and then selfed (or outcrossed and selfed) to obtain seeds homozygous for the nucleic acid construct.

Transformed sorghum tissue or plants can be identified and isolated by screening or selecting for particular traits or activities, e.g., those encoded by a screenable or selectable marker. A transient expression assay for screenable marker activity or expression can be performed at a suitable time after transformation. A suitable time for conducting an assay can be about 1-21 days after transformation, e.g., about 1-14 days, about 1-7 days, or about 1-3 days.

In some cases, transformed sorghum plants can be characterized by the presence of a transferred exogenous nucleic acid comprising a nucleotide sequence encoding a polypeptide, or a transcription product of interest, flanked by at least one T-DNA border, inserted within the genome of the sorghum plant.

Identification of transgenic sorghum plants, produced according to the methods described herein, can include physical and biochemical screening techniques. For example, Southern analysis or PCR amplification for detection of a polynucleotide; northern blots, 51 RNase protection, primer-extension, or RT-PCR amplification for detecting RNA transcripts; enzymatic assays for detecting enzyme or ribozyme activity of polypeptides and polynucleotides; and protein gel electrophoresis, western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides, can be used to identify a transgenic sorghum plant comprising an exogenous nucleic acid. After the presence of a stably incorporated exogenous nucleic acid is confirmed in a regenerated plant, the exogenous nucleic acid can be introduced into other plants using various breeding techniques.

A transgenic sorghum plant made by the methods described herein can be a variety, hybrid or inbred line of grain sorghum (milo), forage sorghum, or a dual purpose sorghum type. Transgenic sweet sorghum types can also be made by such methods. In some embodiments, a suitable species can be a hybrid such as Sorghum×almum, Sorghum×sudangrass or Sorghum×drummondii.

### V. PLANT BREEDING

Fertile transgenic sorghum plants made by methods described herein typically are entered into a plant breeding program. Techniques suitable for use in a sorghum breeding program include, without limitation, backcrossing, mass selection, pedigree breeding, bulk selection, crossing to another population and recurrent selection. These techniques can be used alone or in combination with one or more other techniques in a breeding program. For example, each identified plant can be selfed or crossed a different plant to produce seed that can be germinated to form progeny plants. At least one such progeny plant can be selfed or crossed with a different plant to form a subsequent progeny generation. The breeding program can repeat the steps of selfing or outcrossing for an additional 0 to 5 generations as appropriate in order to achieve the desired uniformity and stability in the resulting plant line, which retains the transgene. In most breeding programs, analysis for the particular polymorphic allele will be carried out in each generation, although analysis can be carried out in alternate generations if desired. Progeny of a transgenic sorghum plant refers to descendants of a particular plant or plant line. Progeny of an instant plant include seeds formed on F₁, F₂, F₃, F₄, F₅, F₆ and subsequent generation plants, seeds formed on BC₁, BC₂, BC₃, and subsequent generation plants, and seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant.

Sorghum plants are bred in most cases by self pollination techniques. With the incorporation of male sterility (either genetic or cytoplasmic) cross pollination breeding techniques can also be utilized. Sorghum has a perfect flower with both male and female parts in the same flower located in the panicle. The flowers are usually in pairs on the panicle branches. Natural pollination occurs in sorghum when anthers (male flowers) open and pollen falls onto receptive stigma (female flowers). Because of the close proximity of male (anthers) and female (stigma) in the panicle, self pollination can be high. Cross pollination may occur when wind or convection currents move pollen from the anthers of one plant to receptive stigma on another plant. Cross pollination is greatly enhanced with incorporation of male sterility which renders male flowers nonviable without affecting the female flowers. Successful pollination in the case of male sterile flowers requires cross pollination.

The development of sorghum hybrids requires the development of homozygous inbred lines, the crossing of these lines, and the evaluation of the crosses. Pedigree breeding methods, and to a lesser extent population breeding methods, are used to develop inbred lines from breeding populations. Breeding programs combine desirable traits from two or more inbred lines into breeding pools from which new inbred lines are developed by selfing and selection of desired phenotypes. The new inbreds are crossed with other inbred lines and the hybrids from these crosses are evaluated to determine which have commercial potential.

Pedigree breeding starts with the crossing of two genotypes, each of which may have one or more desirable characteristics that is lacking in the other or which complement the other. If the two original parents do not provide all of the desired characteristics, other sources can be included in the breeding population. In the pedigree method, superior plants are selfed and selected in successive generations. In the succeeding generations the heterozygous condition gives way to homogeneous lines as a result of self-pollination and selection. Typically, in the pedigree method of breeding five or more generations of selfing and selection is practiced. F₁ to F₂; F₂ to F₃; F₃ to F₄; F₄ to F₅, etc.

Backcrossing can be used to improve an inbred line. Backcrossing transfers a specific desirable trait from one inbred or source to an inbred that lacks that trait. This can be accomplished for example by first crossing a superior inbred (A) (recurrent parent) to a donor inbred (non-recurrent parent), which carries the appropriate genes(s) for the trait in question. The progeny of this cross is then mated back to the superior recurrent parent (A) followed by selection in the resultant progeny for the desired trait to be transferred from the non-recurrent parent. After five or more backcross generations with selection for the desired trait, the progeny will be heterozygous for loci controlling the characteristic being transferred, but will be like the superior parent for most or almost all other genes. The last backcross generation would be selfed to give pure breeding progeny for the gene(s) being transferred.

The production of doubled haploids can also be used for the development of sorghum plants with homozygosity at one or more loci. For example, a transgenic sorghum cultivar can be used as a parent to produce doubled haploid plants. Doubled haploids are produced by the doubling of a set of chromosomes (1 N) from a heterozygous plant to produce a completely homozygous individual. This process obviates the need for generations of selfing needed to obtain a homozygous plant from a heterozygous parent.

A hybrid sorghum variety is the cross of two inbred lines, each of which may have one or more desirable characteristics lacked by the other or which complement the other. The hybrid progeny of the first generation is designated F₁. In the development of hybrids only the F₁ hybrid plants are sought. The hybrid is more vigorous than its inbred parents. This hybrid vigor, or heterosis, can be manifested in many ways, including increased vegetative growth and increased yield.

The development of a hybrid sorghum variety involves five steps: (1) the formation of “restorer” and “non-restorer” germplasm pools; (2) the selection of superior plants from various “restorer” and “non-restorer” germplasm pools; (3) the selfing of the superior plants for several generations to produce a series of inbred lines, which although different from each other, each breed true and are highly uniform; (4) the conversion of inbred lines classified as non-restorers to cytoplasmic male sterile (CMS) forms, and (5) crossing the selected cytoplasmic male sterile (CMS) inbred lines with selected fertile inbred lines (restorer lines) to produce the hybrid progeny (F₁).

Because sorghum is normally a self pollinated plant and because both male and female flowers are in the same panicle, large numbers of hybrid seed can only be produced by using cytoplasmic male sterile (CMS) inbreds. Inbred male sterile lines are developed by converting inbred lines to CMS. This is achieved by transferring the chromosomes of the line to be sterilized into sterile cytoplasm by a series of backcrosses, using a male sterile line as a female parent and the line to be sterilized as the recurrent and pollen parent in all crosses. After conversion to male sterility the line is designated the (A) line. Lines with fertility restoring genes cannot be converted into male sterile A-lines. The original line is designated the (B) line.

Flowers of the CMS inbred are fertilized with pollen from a male fertile inbred carrying genes which restore male fertility in the hybrid (F₁) plants. An important consequence of the homozygosity and homogeneity of the inbred lines is that the hybrid between any two inbreds will always be the same. Once the inbreds that give the best hybrid have been identified, the hybrid seed can be reproduced indefinitely as long as the homogeneity of the inbred parent is maintained.

A single cross hybrid is produced when two inbred lines are crossed to produce the F₁ progeny. Much of the hybrid vigor exhibited by F₁ hybrids is lost in the next generation (F₂). Consequently, seed from hybrid varieties is not typically used for planting stock.

Hybrid sorghum can be produced using wind to move the pollen. Alternating strips of the cytoplasmic male sterile inbred (female) and the male fertile inbred (male) are planted in the same field. Wind moves the pollen shed by the male inbred to receptive stigma on the female. Providing that there is sufficient isolation from sources of foreign sorghum pollen, the stigma of the male sterile inbred (female) will be fertilized only with pollen from the male fertile inbred (male). The resulting seed, born on the male sterile (female) plants is therefore hybrid and will form hybrid plants that have full fertility restored. In some embodiments, if the hybrid sorghum is used as forage or for biomass production, then it may be unnecessary to restore fertility.

Sorghum breeding methods can include the use of genotyping techniques for marker-assisted breeding methods. Suitable genotyping techniques include Isozyme Electrophoresis, Arbitrarily Primed Polymerase Chain Reaction (AP-PCR), DNA Amplification Fingerprinting (DAF), and Sequence Characterized Amplified Regions (SCARs).

Genetic polymorphisms that are useful in such methods include simple sequence repeats (SSRs, or microsatellites), rapid amplification of polymorphic DNA (RAPDs), single nucleotide polymorphisms (SNPs), amplified fragment length polymorphisms (AFLPs) and restriction fragment length polymorphisms (RFLPs). SSR polymorphisms can be identified, for example, by making sequence specific probes and amplifying template DNA from individuals in the population of interest by PCR. For example, PCR techniques can be used to enzymatically amplify a genetic marker associated with a nucleotide sequence conferring a specific trait (e.g., nucleotide sequences described herein). PCR can be used to amplify specific sequences from DNA as well as RNA, including sequences from total genomic DNA or total cellular RNA. When using RNA as a source of template, reverse transcriptase can be used to synthesize complementary DNA (cDNA) strands. Various PCR methods are described, for example, in PCR Primer: A Laboratory Manual, Dieffenbach and Dveksler, eds., Cold Spring Harbor Laboratory Press, 1995.

### VI. ARTICLES OF MANUFACTURE

Transgenic sorghum plants made by the methods described herein have various uses in the agricultural and energy production industries. For example, transgenic sorghum can be used to make animal feed and food products. Such plants are also useful as a feedstock for energy production.

Seeds from sorghum plants described herein can be conditioned and bagged in packaging material by means known in the art to form an article of manufacture. Packaging material such as paper and cloth are well known in the art. A package of seed can have a label, e.g., a tag or label secured to the packaging material, a label printed on the packaging material, or a label inserted within the package, that describes the nature of the seeds therein.

The invention will be further described in the following examples, which do not limit the scope of the invention described in the claims.

### VII. EXAMPLES

### Example 1

### Callus Induction

Inbred sorghum plants (from lines B.Tx635, B.Tx637, B.Tx626, B.Tx2752, and BtX430) were grown in 8 inch pots in Soil Mix, one plant per pot, in a greenhouse under a 16/8 hr day/night photoperiod at 28/24° C. Plants were self-pollinated. Young panicles were harvested at 10-15 days after anthesis. Immature seeds were collected from the panicles, surface-sterilized with 75% EtOH for 2 to 5 minutes followed by 20% commercial bleach and 0.1% Liquinox (surfactant) with shaking for 30 minutes. The tissue was rinsed 5 times with sterile MilliQH₂O. Immature embryos were removed from the panicles under a dissecting microscope using forceps, transferred onto induction medium (SCI) (Table 1), and incubated on SCI medium at 27° C. in the dark in a growth chamber.

TABLE 1
Media for sorghum transformation

| Medium | | | | | | |
|---|---|---|---|---|---|---|
| Components | SCI | SLC | SCS1 | SCS2 | SR tp | SR ttp |
| Basal medium | 1X MS | 1/10 MS | 1X MS | 1X MS | 1X MS | ½ MS |
| CuSO₄•5 H₂O | 0.05 mg/L | — | 0.05 mg/L | 0.05 mg/L | — | — |
| Amino acid | 2 mg/L Glycine | 200 mg/L | 2 mg/L | 2 mg/L Glycine | 50 mg/L L- | 0.2 mg/L |
| | 200 mg/L | cysteine | Glycine | 200 mg/L | Glutamine | Glycine |
| | Asparagine | 30 mg/L | 200 mg/L | Asparagine | | |
| | | glutamine | Asparagine | | | |
| Casamino | 1 g/L | — | 1 g/L | 1 g/L | — | 1 g/L |
| Acid | | | | | | |
| Myo-inositol | 100 mg/L | — | 100 mg/L | 100 mg/L | — | 100 mg/L |
| B5 Vitamins | 1X | 1/10 X | 1X | 1X | 1X | — |
| Thiamine | 5 mg/L | — | 5 mg/L | 5 mg/L | — | 0.5 mg/L |
| Pyridoxine | 1 mg/L | — | 1 mg/L | 1 mg/L | — | 1 mg/L |
| Nicotinic Acid | 1 mg/L | — | 1 mg/L | 1 mg/L | — | 1 mg/L |
| Auxin | 2 mg/L 2,4-D | 2 mg/L 2,4-D | 2 mg/L 2,4-D | 2 mg/L 2,4-D | 0.2 mg/L NAA | 0.5 mg/L NAA |
| Cytokinin | — | 0.5 mg/L BA | — | — | 2 mg/L BA | — |
| Ascorbic Acid | — | 10 mg/L | — | — | — | — |
| Sugar or sugar | 2.5% Sucrose | 3% Maltose | 2.5% Sucrose | 2.5% Sucrose | 1% Maltose | 1.5% Sucrose |
| alcohol | 1% Sorbitol | 1% Glucose | 1% Sorbitol | 1% Sorbitol | 2% Sucrose | |
| Gelling agent | 7 g/L Agarose | — | 7 g/L Agarose | 7 g/L Agarose | 7 g/L Agar | 5 g/L Agarose |
| pH | 5.8 | 5.4 | 5.8 | 5.8 | 5.8 | 5.8 |
| Supplement | — | 300 μM | 125 mg/L | 125 mg/L | 125 mg/L | 125 mg/L |
| | | Aceto- | carbenicillin | carbenicillin | carbenicillin | carbenicillin |
| | | syringone | 50 mg/L | 100 mg/L | 100 mg/L | |
| | | | paramomycin | paramomycin | paramomycin | |

— Not included
SCI = Sorghum Callus Induction medium
SLC = Sorghum Liquid Co-cultivation medium
SCS1 = Sorghum Selection medium 1
SCS2 = Sorghum Selection medium 2
SRtp = Sorghum Regeneration medium
SRttp = Sorghum Rooting medium

The immature sorghum embryos were subcultured on the same medium every two weeks until translucent light yellow embryogenic sorghum calli (EC) had formed. The amount of EC induced on SCI medium was compared to the amount of EC induced on the same medium except that it lacked asparagine and sorbitol. The approximate increase in EC formation on SCI medium is presented in Table 2. These results indicate that culturing immature sorghum embryos on selection medium (containing sorbitol and asparagine) increased embryogenic sorghum callus induction from about 20 to about 35 percent, relative to medium lacking asparagine and sorbitol.

TABLE 2

| | % increase in induction |
|---|---|
| Sorghum line | of embryonic callus |
| B.Tx635 | 35 |
| B.Tx637 | 20 |
| B.Tx627 | 25 |
| B.Tx2752 | 32 |
| B.Tx430 | 25 |

### Example 2

### Sorghum and Agrobacterium Co-Cultivation

Agrobacterium EHA105, containing a binary vector with an NPTII selectable marker construct and a GFP screening construct, was used for co-cultivation. The strain was inoculated into 2 mL YEB liquid medium plus antibiotics, and incubated overnight on a 28° C. shaker. The bacterial culture was re-inoculated into 5 mL YEB plus antibiotics and cultured on a 28° C. shaker overnight. The culture was spun down in a micro-centrifuge and re-suspended in Sorghum Liquid Co-cultivation medium (SLC) (Table 1) in a 50 mL conical tube. The density of the cell suspension was adjusted to between 0.3 and 0.5 OD₆₀₀. The Agrobacterium suspension was kept at 22 to 24° C. in the dark while shaking (150 rpm) until the EC was prepared.

BT×430 EC that were at mid-passage were used for co-cultivation. At about one week during subculture on SCI medium, enough SLC liquid medium was added to a 50 ml conical tube to cover the calli, the tube was heated at 43° C. for 3 minutes and allowed to cool to room temperature.

The SLC was decanted from the tube, and the Agrobacterium suspension was added to the EC in the 50 mL conical tube. The EC and Agrobacterium were incubated on a 22° C. shaker at 110 rpm in the dark for 30 minutes. The Agrobacterium suspension was decanted, and the EC transferred to a sterile Kimwipes® wipe in a petri dish and blotted dry. EC were spread on three sterile Whatman® #1 filter papers moistened with 2.2 mL SLC (moist filter paper) in a 100×20 mm petri dish. Control EC were placed on a single sterile Whatman® #1 filter paper that had been saturated with SLC medium (saturated filter paper). The petri dishes were sealed with plastic wrap and co-cultivated at 25° C. in the dark in a growth chamber.

After 3-4 days of co-cultivation, calli were rinsed with 20-30 mL of 250 mg/L carbenicillin in water. Calli were then blotted briefly with a sterile wipe in a petri dish, transferred onto SCS1 medium (Table 1), and cultured at 28° C. in the dark in a growth chamber for 1 week. After the initial selection period, all calli were transferred to SCS2 medium (Table 1), and incubated at 28° C. in the dark in a growth chamber for two weeks. Calli were then screened for GFP expression. If no GFP expression is observed, calli can be subcultured on SCS2 medium for an additional 14 days and then screened for GFP expression.

Embryogenic calli were screened for GFP expression after 3 days of co-cultivation and 5-7 days on SCS1 medium. Representative results are shown in Table 3 for BTx430, and indicate that co-cultivation on moist filter paper results in 30 to 40% of co-cultivated calli exhibiting GFP expression, as compared to 10 to 20% exhibiting GFP expression when wet filter paper is used. The results indicate that efficiency of transformation of embryogenic sorghum calli can be increased by limiting the moisture during co-cultivation.

TABLE 3

| Co-culture Condition | Total # of calli | # of GFP+ calli | GFP+ calli (%) |
|---|---|---|---|
| Saturated filter paper: | 80 | 14 | 17% |
| | 87 | 8 | 9% |
| Moist filter paper | 75 | 23 | 30% |
| | 51 | 19 | 37% |
| | 28 | 10 | 35% |

### Example 3

### Sorghum Regeneration

Calli from Example 2 that exhibit GFP fluorescence and appear to survive paramomycin selection are transferred to a petri dish containing SRtp medium (Table 1) and cultured 25 days at 28° C. in a growth chamber under a 16/8 hr day/night photoperiod. In some cases, some calli are subcultured for one additional passage under the same conditions.

Plantlets that form are transferred to a Magenta box containing SR ttp medium (Table 1) and incubated at 28° C. in a growth chamber under a 16/8 hr day/night photoperiod for about three weeks until plantlets are well-rooted. When plantlets are about 4 cm or more in height, a leaf sample is collected and PCR is carried out to determine the presence of the NPTII selectable marker or the GFP screenable marker. Well-rooted plants confirmed to be transgenic are transferred to pots containing soil and grown in a greenhouse to maturity. In some cases, transgenic plants are self-pollinated or cross pollinated.

### Example 4

### Callus Induction, Sorghum and Agrobacterium Co-Cultivation, and Sorghum Regeneration

The experiments set forth in Examples 1-3 can be repeated using the media set forth in Table 4. For co-cultivation, petri dishes containing the EC and sterile Whatman® #1 filter papers moistened with 2.2 mL SLC (moist filter paper) were sealed with plastic wrap and co-cultivated at 22° C. in the dark in a growth chamber. After 4 days of co-cultivation, calli were transferred onto SCS1 medium (Table 4), and cultured at 27° C. in the dark in a growth chamber for 14 days. After the initial selection period, all calli were transferred to SCS2 medium (Table 4), and incubated at 28° C. in the dark in a growth chamber for two weeks until transgenic calli were produced. If no transgenic calli are found, calli can be subcultured on SCS2 medium for an additional 14 days and then re-screened. Before regeneration, transformed calli were transferred to SR (Table 4) and incubated at 28° C. in a growth chamber under a 16/8 hr day/night photoperiod for 7 to 14 days.

TABLE 4
Media for sorghum transformation

| Medium | | | | | | | |
|---|---|---|---|---|---|---|---|
| Components | SCI | SR | SLC | SCS1 | SCS2 | SR tp | SR ttp |
| Basal medium | 1X MS | 1X MS | 1/10 MS | 1X MS | 1X MS | 1X MS | ½ MS |
| CuSO₄•5 H₂O | 0.05 mg/L | 0.05 mg/L | — | 0.05 mg/L | 0.05 mg/L | 0.05 mg/L | — |
| Amino acid | 2 mg/L | 2 mg/L | 200 mg/L | 2 mg/L | 2 mg/L | 2 mg/L | 0.2 mg/L |
| | Glycine | Glycine | cysteine | Glycine | Glycine | Glycine | Glycine |
| | 200 mg/L | 200 mg/L | 50 mg/L | 200 mg/L | 200 mg/L | | |
| | Asparagine | Asparagine | glutamine | Asparagine | Asparagine | | |
| | 100 mg/L | 100 mg/L | | 100 mg/L | 100 mg/L | | |
| | Cysteine | L-cysteine | | cysteine | cysteine | | |
| Casamino | 1 g/L | 1 g/L | — | 1 g/L | 1 g/L | 1 g/L | 1 g/L |
| Acid | | | | | | | |
| Myo-inositol | 100 mg/L | 100 mg/L | — | 100 mg/L | 100 mg/L | 100 mg/L | 100 mg/L |
| B5 Vitamins | 1X | 1X | 1/10 X | 1X | 1X | 1X | — |
| Thiamine | 5 mg/L | 5 mg/L | — | 5 mg/L | 5 mg/L | 5 mg/L | 0.5 mg/L |
| Pyridoxine | 1 mg/L | 1 mg/L | — | 1 mg/L | 1 mg/L | 1 mg/L | 1 mg/L |
| Nicotinic Acid | 1 mg/L | 1 mg/L | — | 1 mg/L | 1 mg/L | 1 mg/L | 1 mg/L |
| Auxin | 2 mg/L 2,4-D | 0.5 mg/L NAA | 2 mg/L 2,4-D | 2 mg/L 2,4-D | 2 mg/L 2,4-D | 0.2 mg/L NAA | 0.5 mg/L NAA |
| | | | | 0.5 mg/L NAA | 0.5 mg/L NAA | | |
| Cytokinin | — | 0.5 mg/L BA- | 0.25 mg/L | — | — | 2 mg/L BA | — |
| | | | BA | | | | |
| Ascorbic Acid | — | — | 10 mg/L | — | — | — | — |
| Sugar or sugar | 2.5% Sucrose | 2.5% Sucrose | 3% Maltose | 2.5% Sucrose | 2.5% Sucrose | 2.5% Sucrose | 1.5% Sucrose |
| alcohol | 1% Sorbitol | 1% Sorbitol | 1% Glucose | 1% Sorbitol | 1% Sorbitol | 2.5% Sorbitol | |
| Gelling agent | 7 g/L Agarose | 7 g/L Agarose | — | 7 g/L Agarose | 7 g/L Agarose | 7 g/L Agarose | 7 g/L Agar |
| pH | 5.8 | 5.8 | 5.4 | 5.8 | 5.8 | 5.8 | 5.8 |
| Supplement | — | 125 mg/L | 200 μM | 125 mg/L | 125 mg/L | 125 mg/L | 125 mg/L |
| | | Timentin | Aceto- | Timentin | Timentin | Timentin | Timentin |
| | | | syringone | 50 mg/L | 100 mg/L | 50 mg/L | 50 mg/L |
| | | | | paramomycin | paramomycin | paramomycin | paramomycin |

— Not included
SCI = Sorghum Callus Induction medium
SR = Sorghum resting medium
SLC = Sorghum Liquid Co-cultivation medium
SCS1 = Sorghum Selection medium 1
SCS2 = Sorghum Selection medium 2
SRtp = Sorghum Regeneration medium
SRttp = Sorghum Rooting medium

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for inducing embryogenic sorghum callus, said method comprising
(a) incubating immature sorghum embryos on an induction medium comprising sorbitol and asparagine until embryogenic sorghum callus is formed.

2. The method of claim 1, wherein said sorbitol is present at a concentration of about 0.5% w/v to about 4.0% w/v.

3. The method of claim 1, wherein said asparagine is present at a concentration of about 50 mg/L to about 1,000 mg/L.

4. The method of claim 1, wherein said induction medium further comprises MS basal salts, myo-inositol, pyridoxine, nicotinic acid, and an auxin.

5. The method of claim 4, wherein said induction medium comprises MS basal salts, 0.05 mg/L CuSO₄.5 H₂O, 2 mg/L glycine, 200 mg/L asparagine, 100 mg/L cysteine, 100 mg/L myo-inositol, B5 vitamins, 5 mg/L thiamine, 1 mg/L pyridoxine, 1 mg/L nicotinic acid, 2 mg/L 2,4-D, 2.5% sucrose, 1% sorbitol, and 7 g/L agarose, at pH 5.8.

6. The method of claim 1, wherein said immature sorghum embryos are obtained from a plant selected from the group consisting of a plant of inbred line B.Tx635, inbred line B.Tx637, inbred line B.Tx627, inbred line B.Tx2752, inbred line BtX430 and inbred line C401.

7. A method of making a transformed sorghum cell, said method comprising
(a) incubating immature sorghum embryos on an induction medium comprising sorbitol and asparagine to form embryogenic sorghum callus;
(b) contacting said embryogenic sorghum callus with Agrobacterium in a liquid medium, said Agrobacterium comprising an exogenous nucleic acid whose expression confers resistance to a selection agent;
(c) co-cultivating said embryogenic sorghum callus, after said contacting step, on a co-cultivation medium for a period of about 2 to about 5 days; and
(d) selecting, on a selection medium, for at least one transformed sorghum cell derived from said co-cultivated embryogenic sorghum callus, said selection medium containing an antibiotic that inhibits the growth of said Agrobacterium and a selection agent that inhibits the growth of untransformed sorghum cells, thereby obtaining said transformed sorghum cell.

8. The method of claim 7, said selection medium further comprising sorbitol and asparagine.

9. The method of claim 7, wherein said incubating step comprises two subculturing periods of 14 days each on said induction medium.

10. A method of making a transformed sorghum cell, said method comprising
(a) contacting immature sorghum embryos with Agrobacterium on a liquid medium, said Agrobacterium comprising an exogenous nucleic acid whose expression confers resistance to a selection agent;
(b) co-cultivating said immature embryos, after said contacting step, on a co-cultivation medium for a period of about 2 to about 5 days, said co-cultivating medium comprising sorbitol and asparagine; and
(c) selecting, on a selection medium, for at least one transformed sorghum cell derived from said co-cultivated immature embryos, said selection medium containing an antibiotic that inhibits the growth of said Agrobacterium and a selection agent that inhibits the growth of untransformed sorghum cells, thereby obtaining said transformed sorghum cell.

11. The method of claim 7 or 10, wherein said co-cultivating period is about 3 days.

12. The method of claim 7 or 10, wherein said exogenous nucleic acid is NPTII and said selection agent is paramomycin, or said exogenous nucleic acid is PAT and said selection agent is phosphinothricin.

13. The method of claim 7 or 10, further comprising incubating said transformed sorghum cell in a resting medium for about 7 to about 14 days, said resting medium comprising asparagine, cysteine, an auxin, a cytokinin, and sorbitol.

14. The method of claim 13, wherein said resting medium comprises MS basal salts, 0.05 mg/L CuSO₄.5 H₂O, 2 mg/L glycine, 200 mg/L asparagine, 100 mg/L cysteine, 100 mg/L myo-inositol, B5 vitamins, 5 mg/L thiamine, 1 mg/L pyridoxine, 1 mg/L nicotinic acid, 0.5 mg/L NAA, 2.5% sucrose, 1% sorbitol, and 7 g/L agarose, at pH 5.8.

15. The method of claim 13, further comprising the step of regenerating at least one transformed sorghum plant from said transformed sorghum cell.

16. The method of claim 7 or 10, wherein said immature sorghum embryos are from a plant selected from the group consisting of a plant from inbred line B.Tx635, inbred line B.Tx637, inbred line B.Tx627, inbred line B.Tx2752, inbred line B.Tx430 and inbred line C401.

17. The method of claim 7 or 10, wherein said selecting step comprises selecting a plurality of transformed sorghum cells derived from said co-cultivated immature embryos.

18. The method of claim 17, further comprising the step of regenerating a plurality of transformed sorghum plants from said transformed sorghum cells.

19. The method of claim 7 or 10, wherein said selection medium further comprises one or more auxins.

20. The method of claim 19, wherein said selection medium comprises 2,4-D and NAA.
